(19) United States
(12) Patent Application Publication
(10) Pub. No.: US 2010/0114087 A1
(43) Pub. Date: May 6, 2010

(54) METHODS AND DEVICES FOR TREATING URINARY INCONTINENCE

(76) Inventors: Stuart D. Edwards, Salinas, CA (US); Charles S. Carignan, Boston, MA (US); Simon Thomas, Newark, CA (US)

Correspondence Address: SHAY GLENN LLP, 2755 CAMPUS DRIVE, SUITE 210, SAN MATEO, CA, 94403, US

(21) Appl. No.: 12/494,871

(22) Filed: Jun. 30, 2009

Related U.S. Application Data
(63) Continuation-in-part of application No. 11/651,750, filed on Jan. 10, 2007.
Application No. 11/651,750 is a division of application No. 09/924,935, filed on Aug. 8, 2001, now Pat. No. 7,165,551.
Application No. 09/924,935 is a continuation-in-part of application No. 09/360,599, filed on Jul. 26, 1999, now abandoned.
Application No. 09/360,599 is a continuation of application No. 09/026,085, filed on Feb. 19, 1998, now abandoned.
Application No. 09/026,085 is a continuation of application No. 09/026,296, filed on Feb. 19, 1998, now Pat. No. 6,009,877.

(51) Int. Cl. A61B 18/18

(52) U.S. Cl. 606/33

(57) ABSTRACT

Described herein are devices, systems and methods for treatment of tissue within a lumen of a body. For example, the devices described herein may be used to treat the urethra or gastrointestinal tract, including a sphincter. These devices may provide an expandable element at the distal end of an elongate body and may also include a plurality of electrodes (e.g., needle electrodes) configured to extend from the device and into the tissue to deliver energy to multiple, circumferentially arranged treatment sites. Sufficient energy may be delivered from the device to create a desired tissue effect.

### CROSS REFERENCE TO RELATED APPLICATIONS

This application claims priority as a Continuation-in-Part of pending U.S. patent application Ser. No. 11/420,712, filed on May 26, 2006 and titled “SYSTEM FOR TISSUE ABLATION”, which is a continuation application of U.S. patent application Ser. No. 10/963,025, filed Oct. 12, 2004 (titled “METHODS FOR TREATING THE CARDIA OF THE STOMACH”), which is a divisional of U.S. patent application Ser. No. 10/247,153, filed Sep. 19, 2002 (titled “METHODS FOR TREATING THE CARDIA OF THE STOMACH”), now U.S. Pat. No. 6,872,206, which is a divisional of U.S. patent application Ser. No. 09/304,737, filed May 4, 1999 (titled “STOMACH AND ADJOINING TISSUE REGIONS IN THE ESOPHAGUS”), now U.S. Pat. No. 6,464,697, which is a continuation-in-part of U.S. patent application Ser. No. 09/026,296, filed Feb. 19, 1998 (titled “METHOD FOR TREATING A SPHINCTER”), now U.S. Pat. No. 6,009,877, to which applications we claim priority under 35 U.S.C. §120.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

This invention relates generally to devices and methods for the treatment of tissue within a body, and more specifically to devices and methods that may be used to treat urinary incontinence and esophageal sphincters.

### BACKGROUND OF THE INVENTION

Many disorders and diseases may benefit from the delivery of therapies that apply localized tissue treatment that can be applied through a body orifice. For example, urinary incontinence (UI) and gastroesophageal reflux disease (GERD) may both benefit from the controlled application of energy to tissue associated body regions.

GERD is a common gastroesophageal disorder in which the stomach contents are ejected into the lower esophagus due to a dysfunction of the lower esophageal sphincter (LES). These contents are highly acidic and potentially injurious to the esophagus resulting in a number of possible complications of varying medical severity. The reported incidence of GERD in the U.S. is as high as 10% of the population (Castell D O; Johnston B T: Gastroesophageal Reflux Disease: Current Strategies For Patient Management. Arch Fam Med, 5(4):221-7; (1996 April)).

Acute symptoms of GERD include heartburn, pulmonary disorders and chest pain. On a chronic basis, GERD subjects the esophagus to ulcer formation, or esophagitis and may result in more severe complications including esophageal obstruction, significant blood loss and perforation of the esophagus. Severe esophageal ulcerations occur in 20-30% of patients over age 65. Moreover, GERD causes adenocarcinoma, or cancer of the esophagus, which is increasing in incidence faster than any other cancer (Reynolds J C: Influence Of Pathophysiology, Severity, And Cost On The Medical Management Of Gastroesophageal Reflux Disease. Am J Health Syst Pharm, 53(22 Suppl 3):S5-12 (Nov. 15, 1996)).

One of the possible causes of GERD may be aberrant electrical signals in the LES or cardia of the stomach. Such signals may cause a higher than normal frequency of relaxations of the LES allowing acidic stomach contents to be repeatedly ejected into the esophagus and cause the complications described above. Research has shown that unnatural electrical signals in the stomach and intestine can cause reflux events in those organs (Kelly K A, et al: Duodenal-gastric Reflux and Slowed Gastric Emptying by Electrical Pacing of the Canine Duodenal Pacesetter Potential. Gastroenterology. 1977 March; 72(3): 429-433). In particular, medical research has found that sites of aberrant electrical activity or electrical foci may be responsible for those signals (Karlstrom L H, et al.: Ectopic Jejunal Pacemakers and Enterogastric Reflux after Roux Gastrectomy: Effect Intestinal Pacing. Surgery. 1989 September; 106(3): 486-495). Similar aberrant electrical sites in the heart which cause contractions of the heart muscle to take on life threatening patterns or dysrhythmias can be identified and treated using mapping and ablation devices as described in U.S. Pat. No. 5,509,419. However, there is no current device or associated medical procedure available for the electrical mapping and treatment of aberrant electrical sites in the LES and stomach as a means for treating GERD.

Current drug therapy for GERD includes histamine receptor blockers which reduce stomach acid secretion and other drugs which may completely block stomach acid. However, while pharmacologic agents may provide short term relief, they do not address the underlying cause of LES dysfunction.

Invasive procedures requiring percutaneous introduction of instrumentation into the abdomen exist for the surgical correction of GERD. One such procedure, Nissen fundoplication, involves constructing a new “valve” to support the LES by wrapping the gastric fundus around the lower esophagus. Although the operation has a high rate of success, it is an open abdominal procedure with the usual risks of abdominal surgery including: postoperative infection, herniation at the operative site, internal hemorrhage and perforation of the esophagus or of the cardia. In fact, a recent 10 year, 344 patient study reported the morbidity rate for this procedure to be 17% and mortality 1% (Urschel, J D: Complications Of Antireflux Surgery, Am J Surg 166(1): 68-70; (July 1993)). This rate of complication drives up both the medical cost and convalescence period for the procedure and may exclude portions of certain patient populations (e.g., the elderly and immuno-compromised).

Efforts to perform Nissen fundoplication by less invasive techniques have resulted in the development of laparoscopic Nissen fundoplication. Laparoscopic Nissen fundoplication, reported by Dallemagne et al. Surgical Laparoscopy and Endoscopy, Vol. 1, No. 3, (1991), pp. 138-43 and by Hindler et al. Surgical Laparoscopy and Endoscopy, Vol. 2, No. 3, (1992), pp. 265-272, involves essentially the same steps as Nissen fundoplication with the exception that surgical manipulation is performed through a plurality of surgical cannula introduced using trocars inserted at various positions in the abdomen.

Another attempt to perform fundoplication by a less invasive technique is reported in U.S. Pat. No. 5,088,979. In this procedure an invagination device containing a plurality of needles is inserted transorally into the esophagus with the needles in a retracted position. The needles are extended to engage the esophagus and fold the attached esophagus beyond the gastroesophageal junction. A remotely operated stapling device, introduced percutaneously through an operating channel in the stomach wall, is actuated to fasten the invaginated gastroesophageal junction to the surrounding involuted stomach wall.

Yet another attempt to perform fundoplication by a less invasive technique is reported in U.S. Pat. No. 5,676,674. In this procedure, invagination is done by a jaw-like device and fastening of the invaginated gastroesophageal junction to the fundus of the stomach is done via a transoral approach using a remotely operated fastening device, eliminating the need for an abdominal incision. However, this procedure is still traumatic to the LES and presents the postoperative risks of gastroesophageal leaks, infection and foreign body reaction, the latter two sequela resulting when foreign materials such as surgical staples are implanted in the body.

While the methods reported above are less invasive than an open Nissen fundoplication, some still involve making an incision into the abdomen and hence the increased morbidity and mortality risks and convalescence period associated with abdominal surgery. Others incur the increased risk of infection associated 20 with placing foreign materials into the body. All involve trauma to the LES and the risk of leaks developing at the newly created gastroesophageal junction.

Besides the LES, there are other sphincters in the body which, if not functionally properly can cause disease states or otherwise adversely affect the lifestyle of the patient. Reduced muscle tone or otherwise aberrant relaxation of sphincters can result in a laxity of tightness disease states including, but not limited to, urinary incontinence.

Urinary incontinence arises in both women and men with varying degrees of severity, and from different causes. In men, the condition occurs most often as a result of prostatectomies which result in mechanical damage to the sphincter. In women, the condition typically arises after pregnancy where musculoskeletal damage has occurred as a result of inelastic stretching of the structures which support the genitourinary tract. Specifically, pregnancy can result in inelastic stretching of the pelvic floor, the external sphincter, and most often, to the tissue structures which support the bladder and bladder neck region. In each of these cases, urinary leakage typically occurs when a patient's intra-abdominal pressure increases as a result of stress, e.g. coughing, sneezing, laughing, exercise, or the like.

Treatment of urinary incontinence can take a variety of forms. Most simply, the patient can wear absorptive devices or clothing, which is often sufficient for minor leakage events. Alternatively or additionally, patients may undertake exercises intended to strengthen the muscles in the pelvic region, or may attempt behavior modification intended to reduce the incidence of urinary leakage.

In cases where such non-interventional approaches are inadequate or unacceptable, the patient may undergo surgery to correct the problem. A variety of procedures have been developed to correct urinary incontinence in women. Several of these procedures are specifically intended to support the bladder neck region. For example, sutures, straps, or other artificial structures are often looped around the bladder neck and affixed to the pelvis, the endopelvic fascia, the ligaments which support the bladder, or the like. Other procedures involve surgical injections of bulking agents, inflatable balloons, or other elements to mechanically support the bladder neck.

Each of these procedures has associated shortcomings. Surgical operations which involve suturing of the tissue structures supporting the urethra or bladder neck region require great skill and care to achieve the proper level of artificial support. In other words, it is necessary to occlude or support the tissues sufficiently to inhibit urinary leakage, but not so much that intentional voiding is made difficult or impossible. Balloons and other bulking agents which have been inserted can migrate or be absorbed by the body. The presence of such inserts can also be a source of urinary tract infections. Therefore, it would be desirable to provide an improved therapy for urinary incontinence.

A variety of other problems can arise when the support tissues of the body have excessive length. Excessive length of the pelvic support tissues (particularly the ligaments and fascia of the pelvic area) can lead to a variety of ailments including, for example, cystocele, in which a portion of the bladder protrudes into the vagina. Excessive length of the tissues supporting the breast may cause the breasts to sag. Many hernias are the result of a strained, torn, and/or distended containing tissue, which allows some other tissue or organ to protrude beyond its contained position. Cosmetic surgeries are also often performed to decrease the length of support tissues. For example, abdominoplasty (often called a “tummy tuck”) is often performed to decrease the circumference of the abdominal wall. The distortion of these support tissues may be due to strain, advanced age, congenital predisposition, or the like.

Unfortunately, many support tissues are difficult to access, and their tough, fibrous nature can complicate their repair. As a result, the therapies now used to improve or enhance the support provided by the ligaments and fascia of the body often involve quite invasive surgical procedures.

Thus, it would be desirable to provide improved devices, methods, and systems for treating disorders such as UI and GERD. In particular, it would be useful to provide methods, devices and systems for treating sphincters, fascia, tendons, and the other support tissues of the body. It would be particularly desirable to provide minimally invasive therapies for these support tissues, especially for the treatment of urinary incontinence. It would further be desirable to provide treatment methods which made use of the existing support structures of the body, rather than depending on the specific length of an artificial support structure.

### SUMMARY OF THE INVENTION

The present invention relates to methods, device and systems for delivery of energy (and particularly RF energy) to a treatment site such as a boy lumen like the urethra, esophogeous or the like. Accordingly, an object of the present invention is to provide a method to treat a lumen using methods, devices and system for forming an annular pattern of circumferentially spaced-apart regions. These devices, methods and systems may be applied to treat sphincters and reduce a frequency of sphincter relaxation. Treatment of sphincters is one sub-set of the treatment methods that may be applied by the devices and systems described herein.

In general, the treatment methods described herein may be used to modify the target tissue. For example, the tissue may be lesioned. A lesion may be a change in the structure of the tissue, including a change in the elasticity of the tissue, shrinkage of the tissue, or in some cases necrosis of the tissue. For example, the application of energy to the target tissue may heat the tissue (e.g., fascia and other collagenated support tissues), which may cause them to contract or change elasticity. In some variations, the tissue may be lesioned or modified without substantial necrosis of adjacent tissues. In some variations, the tissue may be necrosed. Thus, in some variations, an object of the invention is to provide a method to create controlled cell necrosis in a sphincter tissue underlying a sphincter mucosal layer. In some variations, an object of the present invention is to modify the elasticity of the tissue without substantial necrosis of the tissues.

As described herein, the energy is provided using a treatment device that is elongate (e.g., for insertion into a body lumen) and includes an expandable region (e.g., anchor) and one or more electrodes such as needle electrodes. These devices may be configured for extension of the electrodes from the device (e.g., the shaft of the device or the expandable member) into the target tissue to from a circumferential pattern of treatment sites by applying RF energy from the electrodes. In some variations a cooling fluid (e.g., water ,saline or glycine) may be applied to or near the treatment sites to control the heating of the tissue or limit the heating of adjacent tissues that are not being directly treated.

For example, described herein are devices for delivering energy to a treatment site in an annular pattern of circumferentially spaced-apart regions in the tissue of a body. The treatment site may be a region of the esophagus (e.g., a region having a sphincter) or a region of the urethra. In some variations, the device includes: an elongate shaft; an expandable member near the distal end of the elongate shaft configured to secure the treatment device within the body; a plurality of needle electrodes configured to extend from the device to contact tissue in an annular array that is circumferentially spaced about the axis, each electrode configured to transmit radio frequency energy to the tissue; and a temperature sensor for sensing the temperature of the tissue at a treatment site.

In some variations, the device may also include a fluid channel for delivery of a fluid from the device. The fluid channel may be configured to apply a cooling fluid from a proximally located reservoir. The fluid channel may pass through the elongate shaft. In some variations, the fluid channel passes through each of the needle electrodes for delivery of a fluid out of the needle electrodes.

The devices described herein may also include a handle at the proximal end of the device configured to manipulate the device. The handle may include an attachment to a controller and/or power supply, one or more connectors to a fluid source, and/or a connector to a source of vacuum or pressurized gas (e.g., to inflate the expandable member).

The expandable member may comprise a basket assembly. For example, the basket assembly may include a plurality of arms that can be extended from the elongate body. In some variations, the expandable member comprises a balloon.

The devices described herein may include a single temperature sensor or a plurality of temperature sensors.

In some variations, the devices may also include a distal cap. The distal cap may be a protective cap (e.g., an atraumatic cap) that does not penetrate tissue. The elongate body may also be flexible.

Also described herein are systems for delivering energy to a treatment site in an annular pattern of circumferentially spaced-apart regions in the tissue of a body. These systems may include any of the treatment devices described, as well as one or more power (energy) sources and/or controllers. For example, a system may include: a treatment device, a power source and a controller. The treatment device may include: an elongate shaft; an expandable member near the distal end of the elongate shaft; a plurality of needle electrodes configured to penetrate the tissue in an annular array that is circumferentially spaced about the axis of the elongate shaft, each electrode configured to transmit radio frequency energy to the tissue; and a temperature sensor. The power source may be configured to provide radio frequency energy to treatment device. The controller may be configured to supply radio frequency energy from the power source at to the needle electrodes to treat the tissue in an annular pattern of individual, circumferentially spaced-apart tissue regions, the controller being coupled to the temperature sensor to control the power supplied by the power source to the electrodes.

As mentioned, the treatment device may include a fluid channel for delivery of a fluid from the treatment device, a handle at the proximal end of the treatment device configured to manipulate the treatment device, or the like. The expandable member of the treatment device may be a basket assembly and/or a balloon.

In some variations, the plurality of needle electrodes may be configured to controllably extend from the shaft. The needle electrodes may also be configured to extend from (or through) the expandable element.

The temperature sensor may be located adjacent to the needle electrodes and configured to sense the temperature of the tissue at a treatment site.

Also described herein are methods of delivering energy to a tissue in an annular pattern of circumferentially spaced-apart treatment sites in the tissue of a patient's body. As mentioned, these methods may be applied to a body lumen, including a urethra or an esophagus. For example, the method may include the steps of: providing a treatment device having a plurality of tissue-piercing needle electrodes configured to extend from the treatment device and an expandable member at the distal end of the treatment device; advancing the treatment device distally within a lumen in a patient's body to a treatment site; expanding the expandable member; extending the tissue-piercing needle electrodes from the treatment device into the tissue; and delivering RF energy from the needle electrodes in an annular pattern of individual, circumferentially spaced-apart tissue regions.

The method may also include the step of pulling proximally upon the treatment device until resistance to the pulling is encountered before extending the tissue-piercing needle electrodes. The method may also include the step of introducing a cooling fluid to cool tissue adjacent to the treatment site.

In some variations, the method further includes sensing a tissue temperature, and controlling delivery of RF energy based, at least in part, upon the sensed tissue temperature condition.

The step of advancing the treatment device distally may include advancing the treatment device within a patient's urethra. The step of delivering RF energy may include producing a plurality of submucosal lesions in an annular pattern of individual, circumferentially spaced-apart tissue regions. This may result in at least partially denaturing collagen in this region.

In one example, described herein is a method of delivering energy to a tissue in an annular pattern of circumferentially spaced-apart treatment sites in the tissue in a patient's urethra, including the steps of: providing a treatment device having a plurality of tissue-piercing needle electrodes configured to extend from the treatment device and an expandable member at the distal end of the treatment device; advancing the treatment device distally within a patient's urethra; expanding the expandable member within the patient's bladder; extending at least one tissue-piercing needle electrode from the treatment device into the tissue; and delivering RF energy from the needle electrodes in an annular pattern of individual, circumferentially spaced-apart tissue regions.

### BRIEF DESCRIPTION OF THE DRAWINGS

FIG. 1 is an illustrated lateral view of the upper GI tract including the esophagus and lower esophageal sphincter and the positioning of a treatment apparatus in the lower esophageal sphincter.

FIG. 2 is a lateral view of a treatment apparatus illustrating an energy delivery device, power supply and expansion member in an expanded and contracted state.

FIG. 3 depicts a lateral view of an apparatus that illustrates components on the flexible shaft including a proximal fitting, connections and proximal and distal shaft segments.

FIG. 4 illustrates a lateral view of a basket assembly.

FIG. 5A is a lateral view of the basket assembly that illustrates the range of camber in the basket assembly.

FIG. 5B is a perspective view illustrating a balloon coupled to the basket assembly.

FIG. 6A is a lateral view of the junction between the basket arms and the shaft illustrating the pathway used for advancement of the movable wire or the delivery of fluids.

FIG. 6B is a frontal view of a basket arm in an alternative embodiment of an apparatus, illustrating a track in the arm used to advance the movable wire.

FIG. 7 is a cross-sectional view of a section of the basket arm illustrating stepped and tapered sections in basket arm apertures.

FIG. 8 is a lateral view of the basket assembly illustrating the placement of the radial supporting member.

FIG. 9A is a lateral view of the sphincter treatment apparatus, illustrating the mechanism used in one embodiment to increase the camber of the basket assembly.

FIG. 9B is a similar view to 9A showing the basket assembly in an increased state of camber.

FIG. 10 is a lateral view of a sphincter treatment apparatus, illustrating the deflection mechanism.

FIG. 11 is a lateral view illustrating the use of electrolytic solution to create an enhanced RF electrode.

FIG. 12 is a lateral view of the basket assembly illustrating the use of needle electrodes.

FIG. 13 is a lateral view illustrating the use of an insulation segment on the needle electrode to protect an area of tissue from RF energy.

FIG. 14 is a lateral view illustrating the placement of needle electrodes into the sphincter wall by expansion of the basket assembly.

FIG. 15 is a lateral view illustrating placement of needle electrodes into the sphincter wall by advancement of an electrode delivery member out of apertures in the basket arms.

FIG. 16 is a cross sectional view illustrating the configuration of a basket arm aperture used to select and maintain a penetration angle of the needle electrode into the sphincter wall.

FIG. 17 is a lateral view illustrating placement of needle electrodes into the sphincter wall by advancement of an electrode delivery member directly out of the distal end of the shaft.

FIG. 18A is a lateral view illustrating a radial distribution of electrodes on the expansion device useful with the method of the present invention.

FIG. 18B is a lateral view illustrating a longitudinal distribution of electrodes on the expansion device.

FIG. 18C is a lateral view illustrating a spiral distribution of electrodes on the expansion device.

FIG. 19 is a flow chart illustrating the sphincter treatment method.

FIG. 20 is a lateral view of sphincter smooth muscle tissue illustrating electromagnetic foci and pathways for the origination and conduction of aberrant electrical signals in the smooth muscle of the lower esophageal sphincter or other tissue.

FIG. 21 is a lateral view of a sphincter wall illustrating the infiltration of tissue healing cells into a lesion in the smooth tissue of a sphincter following treatment with the sphincter treatment apparatus.

FIG. 22 is a view similar to that of FIG. 21 illustrating shrinkage of the lesion site caused by cell infiltration.

FIG. 23 is a lateral view of the esophageal wall illustrating the preferred placement of lesions in the smooth muscle layer of an esophageal sphincter.

FIG. 24 is a lateral view illustrating the ultrasound transducer, ultrasound lens and power source of an embodiment of a treatment device.

FIGS. 25A-D are lateral views of the sphincter wall illustrating various patterns of lesions created by a treatment device.

FIG. 26 is a lateral view of the sphincter wall illustrating the delivery of cooling fluid to the electrode-tissue interface and the creation of cooling zones.

FIG. 27 depicts the flow path, fluid connections and control unit employed to deliver fluid to the electrode-tissue interface.

FIG. 28 depicts the flow path, fluid connections and control unit employed to deliver fluid to the RF electrodes.

FIG. 29 is an enlarged lateral view illustrating the placement of sensors on the expansion device or basket assembly.

FIG. 30 depicts a block diagram of the feedback control system that can be used with the treatment apparatus.

FIG. 31 depicts a block diagram of an analog amplifier, analog multiplexer and microprocessor used with the feedback control system of FIG. 30.

FIG. 32 depicts a block diagram of the operations performed in the feedback control system depicted in FIG. 30.

FIG. 33 is a block diagram of another variation of a method of treating a body lumen.

### DETAILED DESCRIPTION OF THE INVENTION

The devices described herein may be referred to treatment devices, as treatment apparatus, as sphincter treatment devices, as sphincter treatment apparatus, or as urethra treatment devices. Any of these devices may be used to treat a body lumen, including, but not limited to the urethra and the gastrointestinal tract. In general, these devices include an elongate body, an expandable distal region, and a plurality of energy-emitting regions (e.g., needle electrodes).

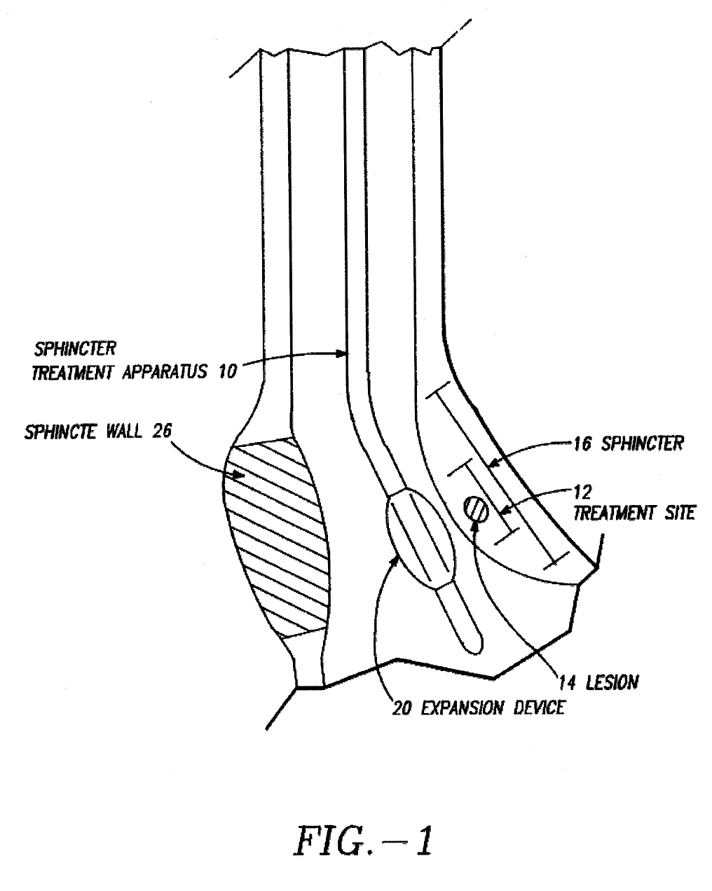

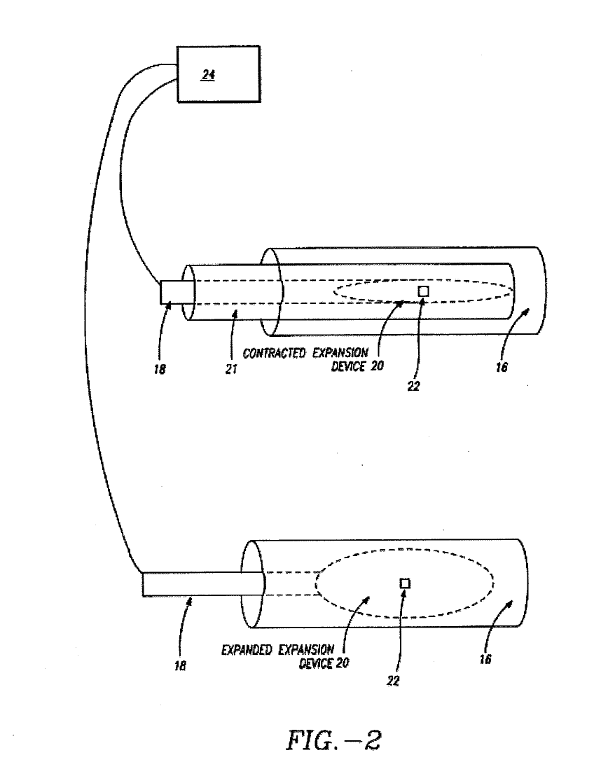

For example, referring now to FIGS. 1 and 2, one embodiment of treatment apparatus 10 that is used to deliver energy to a treatment site 12 to produce lesions 14 in lumen, including lumen having a sphincter 16, such as the lower esophageal sphincter (LES), comprises a flexible elongate shaft 18, also called shaft 18, coupled to a expansion device 20, in turn coupled with one or more energy delivery devices 22. Energy delivery devices 22 are configured to be coupled to a power source 24. The expansion device 20 is configured to be positionable in a sphincter 16 such as the LES or adjacent anatomical structure, such as the cardia of the stomach. Expansion device 20 is further configured to facilitate the positioning of energy delivery devices 22 to a selectable depth in a sphincter wall 26 or adjoining anatomical structure. Expansion device 20 has a central longitudinal axis 28 and is moveable between contracted and expanded positions substantially there along. This can be accomplished by a ratchet mechanism as is known to those skilled in the art. At least portions of sphincter treatment apparatus 10 may be sufficiently radiopaque in order to be visible under fluoroscopy and/or sufficiently echogenic to be visible under ultrasonography. Also as will be discussed herein, sphincter treatment apparatus 10 can include visualization capability including, but not limited to, a viewing scope, an expanded eyepiece, fiber optics, video imaging and the like.

Referring to FIG. 2, shaft 18 is configured to be coupled to expansion device 20 and has sufficient length to position expansion device 20 in the LES and/or stomach using a transoral approach. Typical lengths for shaft 18 include, but are not limited to, a range of 40-180 cms. In various embodiments, shaft 18 is flexible, articulated and steerable and can contain fiber optics (including illumination and imaging fibers, fluid and gas paths, and sensor and electronic cabling. In one embodiment, shaft 18 can be a multi-lumen catheter, as is well known to those skilled in the art. In another embodiment, an introducing member 21, also called an introducer, is used to introduce sphincter treatment apparatus 10 into the LES. Introducer 21 can also function as a sheath for expansion device 20 to keep it in a nondeployed or contracted state during introduction into the LES. In various embodiments, introducer 21 is flexible, articulated and steerable and contains a continuous lumen of sufficient diameter to allow the advancement of sphincter treatment apparatus 10. Typical diameters for introducer 21 include 0.1 to 2 inches, while typical lengths include 40-180 cms. Suitable materials for introducer 21 include coil-reinforced plastic tubing as is well known to those skilled in the art.

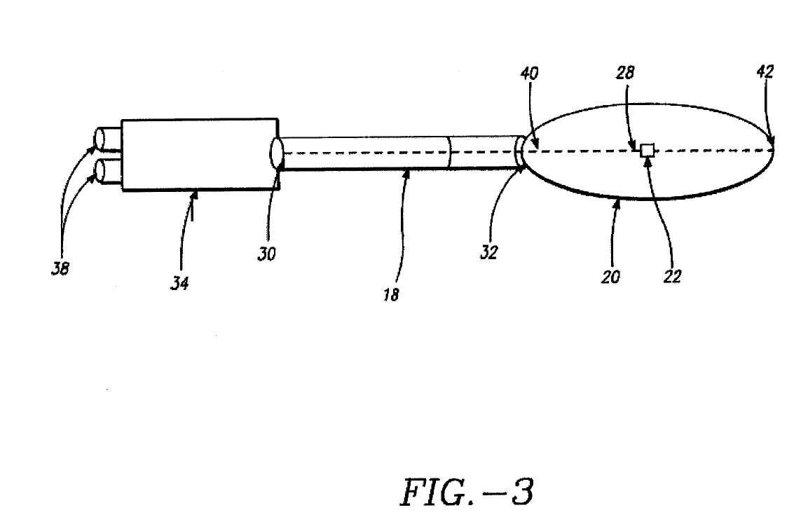

Referring now to FIG. 3, the flexible elongate shaft 18 is circular in cross section and has proximal and distal extremities (also called ends) 30 and 32. Shaft 18 may also be coupled at its proximal end 32 to a proximal fitting 34, also called a handle, used by the physician to manipulate treatment apparatus 10 to reach treatment site 12. Shaft 18 may have one or more lumens 36 that extend the full length of shaft 18, or part way from shaft proximal end 30 to shaft distal end 32. Lumens 36 may be used as paths for catheters, guide wires, pull wires, insulated wires and cabling, fluid and optical fibers. Lumens 36 are connected to and/or accessed by connections 38 on or adjacent to proximal fitting 34. Connections 38 can include luer-lock, lemo connector, swage and other mechanical varieties well known to those skilled in the art. Connections 38 can also include optical/video connections which allow optical and electronic coupling of optical fibers and/or viewing scopes to illuminating sources, eye pieces and video monitors. In various embodiments, shaft 18 may stop at the proximal extremity 40 of expansion device 20 or extend to, or past, the distal extremity 42 of expansion device 20. Suitable materials for shaft 18 include, but are not limited to, polyethylenes, polyurethanes and other medical plastics known to those skilled in the art.

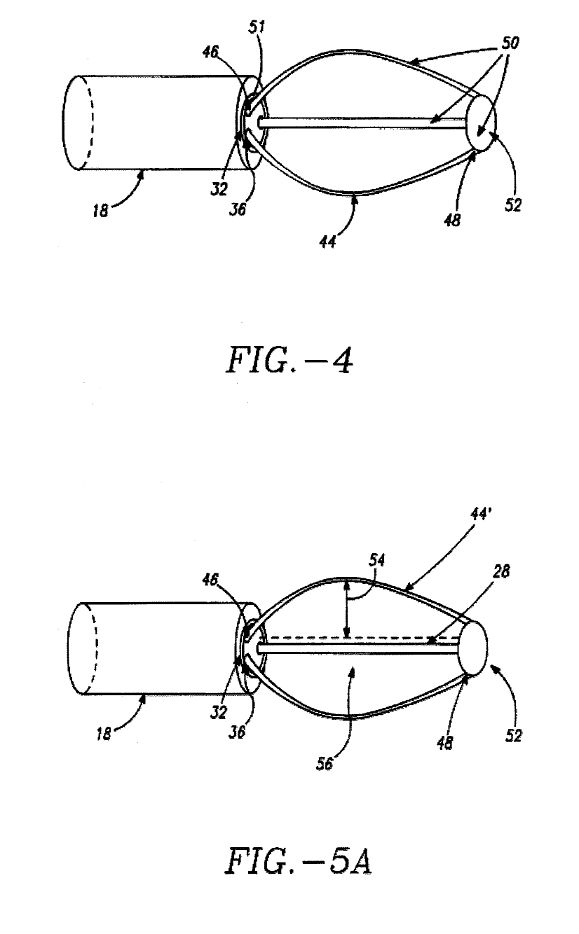

Referring now to FIG. 4, in one embodiment of the present invention, expansion device 20 comprises one or more elongated arms 44 that are joined at their proximal ends 46 and distal ends 48 to form a basket assembly 50. Proximal arm end 46 is attached to a supporting structure, which can be the distal end 32 of shaft 18 or a proximal cap 51. Likewise, distal arm end 48 is also attached to a supporting structure which can be a basket cap 52 or shaft 18. Attached arms 44 may form a variety of geometric shapes including, but not limited to, curved, rectangular, trapezoidal and triangular. Arms 44 can have a variety of cross sectional geometries including, but not limited to, circular, rectangular and crescent-shaped. Also, arms 44 are of a sufficient number (two or more), and have sufficient spring force (0.01 to 0.5 lbs. force) so as to collectively exert adequate force on sphincter wall 26 to sufficiently open and efface the folds of sphincter 16 to allow treatment with sphincter treatment apparatus 10, while preventing herniation of sphincter wall 26 into the spaces 53 between arms 44. Suitable materials for arms 44 include, but are not limited to, spring steel, stainless steel, superelastic shape memory metals such as nitinol or wire reinforced plastic tubing as is well known to those skilled in the art.

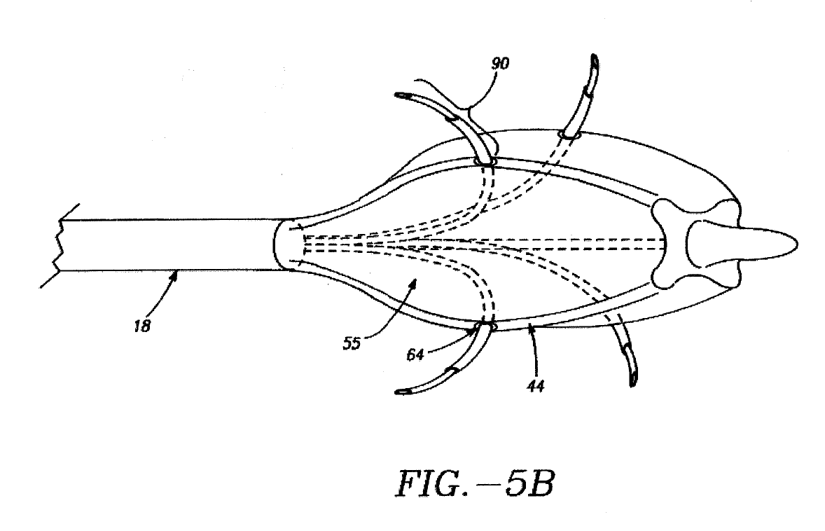

Referring to FIG. 5A, arms 44 can have an outwardly bowed shaped memory for expanding the basket assembly into engagement with a lumen or body region, including a subject's bladder, urethra, or sphincter wall 26 with the amount of bowing, or camber 54 being selectable from a range 0 to 2 inches from longitudinal axis 28 of basket assembly 50. For the case of a curve-shaped arm 44′, expanded arms 44′ are circumferentially and symmetrically spaced-apart.

In another embodiment shown in FIG. 5B, an expandable member 55, which can be a balloon, is coupled to an interior or exterior of basket assembly 50. Balloon 55 is also coupled to and inflated by lumen 36 using gas or liquid. In various other embodiments (not shown), arms 44 may be asymmetrically spaced and/or distributed on an arc less than 360° . Also, arms 44 may be preshaped at time of manufacture or shaped by the physician. In some variations the expandable member includes only a balloon, and does not also include a basket.

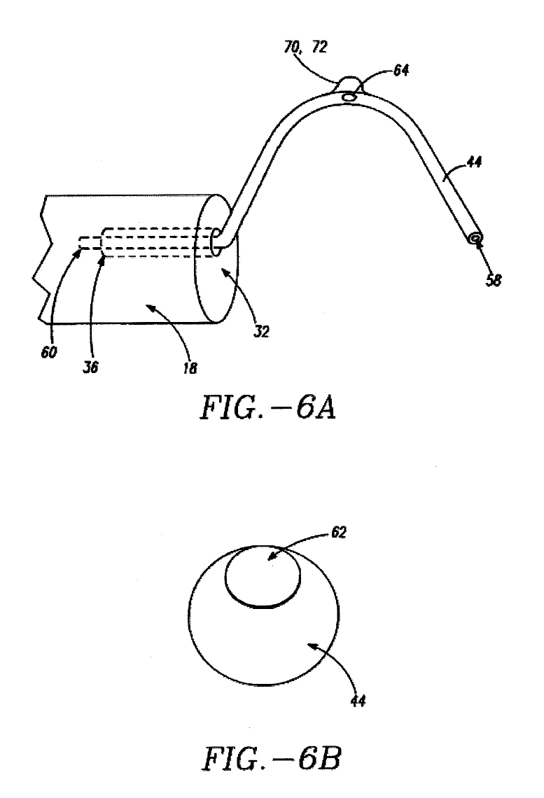

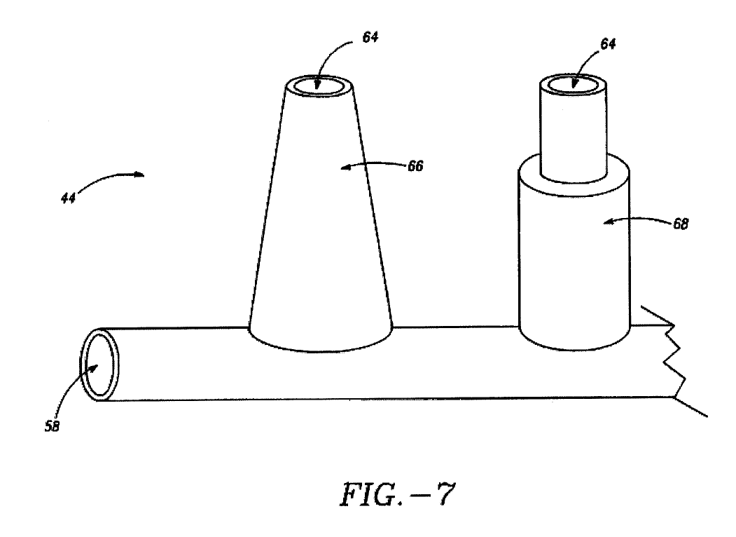

Referring now to FIG. 6A, arms 44 may also be solid or hollow with a continuous lumen 58 that may be coupled with shaft lumens 36. These coupled lumens provide a path for the delivery of a fluid or electrode delivery member 60 from shaft 18 to any point on basket assembly 50. In various embodiments electrode delivery member 60 can be an insulated wire, an insulated guide wire, a plastic-coated stainless steel hypotube with internal wiring or a plastic catheter with internal wiring, all of which are known to those skilled in the art. As shown in FIG. 6B, arms 44 may also have a partially open channel 62, also called a track 62, that functions as a guide track for electrode delivery member 60. Referring back to FIG. 6A, arms 44 may have one or more apertures 64 at any point along their length that permit the controlled placement of energy delivery devices 22 at or into sphincter wall 26. Referring now to FIG. 7, apertures 64 may have tapered sections 66 or stepped sections 68 in all or part of their length, that are used to control the penetration depth of energy delivery devices 22 into sphincter wall 26. Referring back to FIG. 6A, apertures 64 in combination with arm lumens 58 and shaft lumens 36 may be used for the delivery of cooling solution 70 or electrolytic solution 72 to treatment site 12 as described herein. Additionally, arms 44 can also carry a plurality of longitudinally spaced apart radiopaque and or echogenic markers or traces, not shown in the drawings, formed of suitable materials to permit viewing of basket assembly 50 via fluoroscopy or ultrasonography. Suitable radiopaque materials include platinum or gold, while suitable echogenic materials include gas filled micro-particles as described in U.S. Pat. Nos. 5,688,490 and 5,205,287. Arms 44 may also be color-coded to facilitate their identification via visual medical imaging methods and equipment, such as endoscopic methods, which are well known to those skilled in the art.

In another embodiment of the present invention, a supporting member 74 is attached to two or more arms 44. Supporting member 74, also called a strut, can be attached to arms 44 along a circumference of basket assembly 50 as shown in FIG. 8. Apertures 64 can extend through radial supporting member 74 in one or more places. Radial supporting member 74 serves the following functions: i) facilitates opening and effacement of the folds of sphincter 16, ii) enhances contact of Apertures 64 with sphincter wall 26; and, iii) reduces or prevents the tendency of arms 44 to bunch up. The cross sectional geometry of radial supporting member 74 can be rectangular or circular, though it will be appreciated that other geometries are equally suitable.

In one embodiment shown in FIG. 9, arms 44 are attached to basket cap 52 that in turn, moves freely over shaft 18, but is stopped distally by shaft cap 78. One or more pull wires 80 are attached to basket cap 52 and also to a movable fitting 82 in proximal fitting 34 of sphincter treatment apparatus 10. When pull wire 80 is pulled back by movable fitting 82, the camber 54 of basket assembly 50 increases to 54′, increasing the force and the amount of contact applied by basket assembly 50 to sphincter wall 26 or an adjoining structure. Basket assembly 50 can also be deflected from side to side using deflection mechanism 80. This allows the physician to remotely point and steer the basket assembly within the body. In one embodiment shown in FIG. 10, deflection mechanism 84 includes a second pull wire 80′ attached to shaft cap 78 and also to a movable slide 86 integral to proximal fitting 34.

Turning now to a discussion of energy delivery, suitable power sources 24 and energy delivery devices 22 that can be employed in one or more embodiments of the invention include: (i) a radio-frequency (RF) source coupled to an RF electrode, (ii) a coherent source of light coupled to an optical fiber, (iii) an incoherent light source coupled to an optical fiber, (iv) a heated fluid coupled to a catheter with a closed channel configured to receive the heated fluid, (v) a heated fluid coupled to a catheter with an open channel configured to receive the heated fluid, (vi) a cooled fluid coupled to a catheter with a closed channel configured to receive the cooled fluid, (vii) a cooled fluid coupled to a catheter with an open channel configured to receive the cooled fluid, (viii) a cryogenic fluid, (ix) a resistive heating source, (x) a microwave source providing energy from 915 MHz to 2.45 GHz and coupled to a microwave antenna, (xi) an ultrasound power source coupled to an ultrasound emitter, wherein the ultrasound power source produces energy in the range of 300 KHZ to 3 GHz, or (xii) a microwave source. For ease of discussion for the remainder of this application, the power source utilized is an RF source and energy delivery device 22 is one or more RF electrodes 88, also described as electrodes 88. However, all of the other herein mentioned power sources and energy delivery devices are equally applicable to treatment apparatus 10.

For the case of RF energy, RF electrode 88 may operated in either bipolar or monopolar mode with a ground pad electrode. In a monopolar mode of delivering RF energy, a single electrode 88 is used in combination with an indifferent electrode patch that is applied to the body to form the other electrical contact and complete an electrical circuit. Bipolar operation is possible when two or more electrodes 88 are used. Multiple electrodes 88 may be used. These electrodes may be cooled as described herein. Electrodes 88 can be attached to electrode delivery member 60 by the use of soldering methods which are well known to those skilled in the art. Suitable solders include Megabond Solder supplied by the Megatrode Corporation (Milwaukee, Wis.).

Suitable electrolytic solutions 72 include saline, solutions of calcium salts, potassium salts, and the like. Electrolytic solutions 72 may enhance the electrical conductivity of the targeted tissue at the treatment site 12. When a highly conductive fluid such as electrolytic solution 72 is infused into tissue the electrical resistance of the infused tissue is reduced, in turn, increasing the electrical conductivity of the infused tissue. As a result, there will be little tendency for tissue surrounding electrode 88 to desiccate (a condition described herein that increases the electrical resistance of tissue) resulting in a large increase in the capacity of the tissue to carry RF energy. Referring to FIG. 11, a zone of tissue which has been heavily infused with a concentrated electrolytic solution 72 can become so conductive as to actually act as an enhanced electrode 88′. The effect of enhanced electrode 88′ is to increase the amount of current that can be conducted to the treatment site 12, making it possible to heat a much greater volume of tissue in a given time period.

Also when the power source is RF, power source 24, which will now be referred to as RF power source 24, may have multiple channels, delivering separately modulated power to each electrode 88. This reduces preferential heating that occurs when more energy is delivered to a zone of greater conductivity and less heating occurs around electrodes 88 which are placed into less conductive tissue. If the level of tissue hydration or the blood infusion rate in the tissue is uniform, a single channel RF power source 24 may be used to provide power for generation of lesions 14 relatively uniform in size.

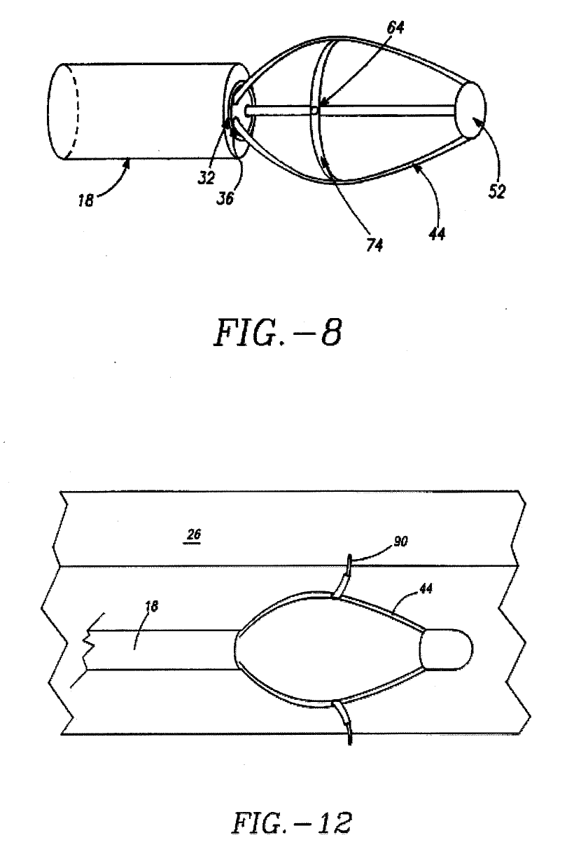

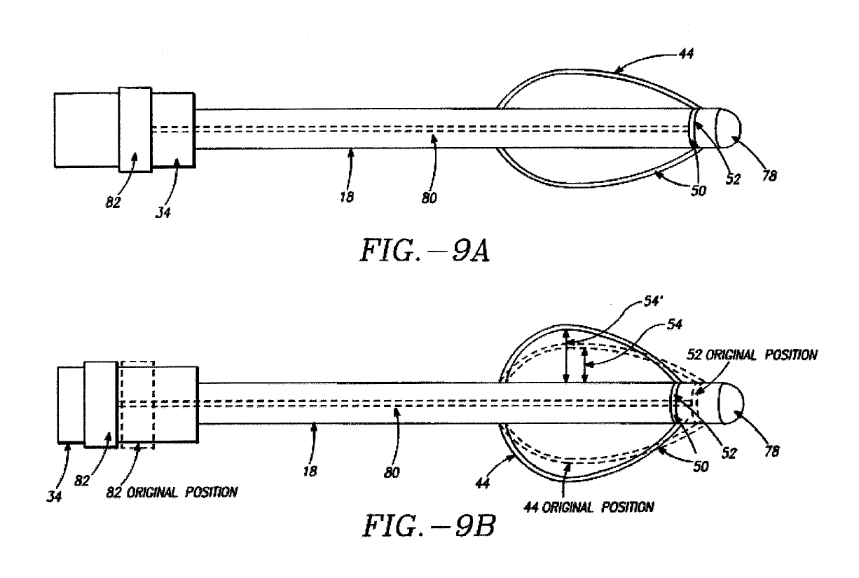

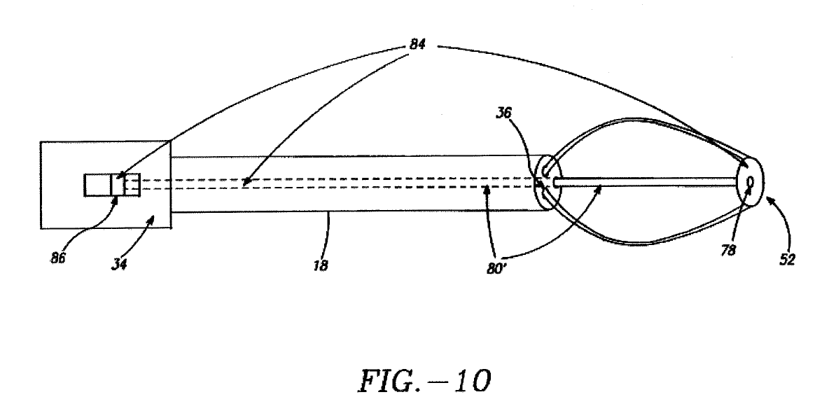

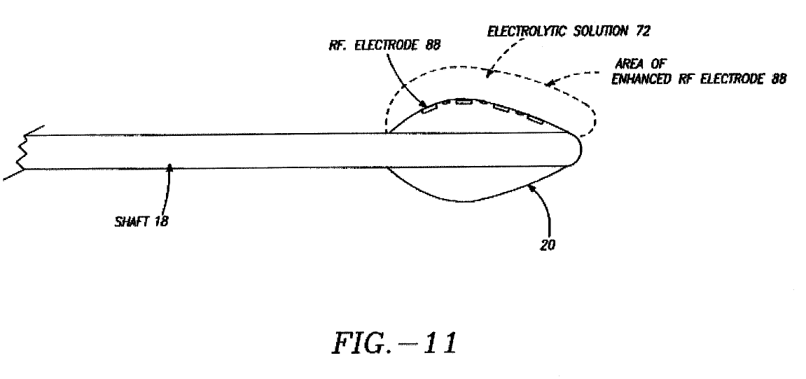

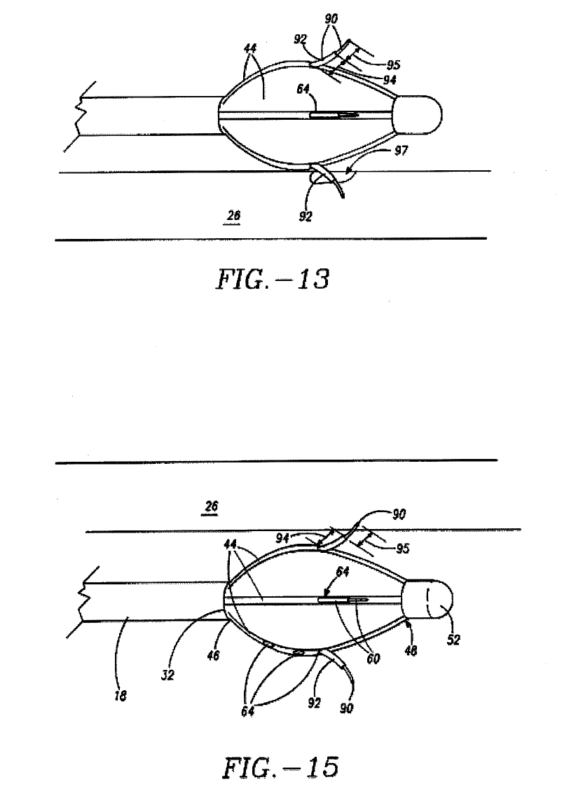

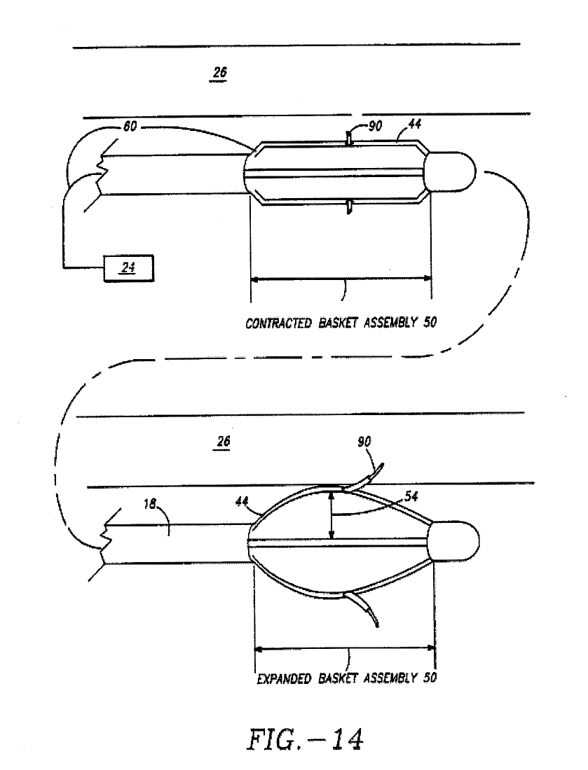

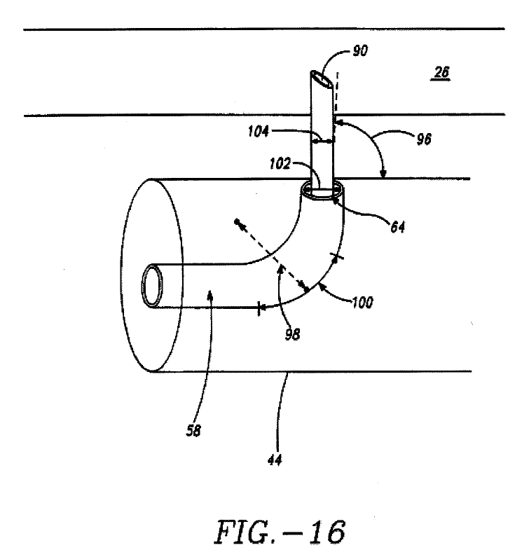

Electrodes 88 can have a variety of shapes and sizes. Possible shapes include, but are not limited to, circular, rectangular, conical and pyramidal. Electrode surfaces can be smooth or textured and concave or convex. The conductive surface area of electrode 88 can range from 0.1 mm² to 100 cm². It will be appreciated that other geometries and surface areas may be equally suitable. In one embodiment, electrodes 88 can be in the shape of needles and of sufficient sharpness and length to penetrate into the smooth muscle of the esophageal wall, sphincter 16 or other anatomical structure. In this embodiment shown in FIGS. 12 and 13, needle electrodes 90 are attached to arms 44 and have an insulating layer 92, covering an insulated segment 94 except for an exposed segment 95. For purposes of this disclosure, an insulator or insulation layer is a barrier to either thermal, RF or electrical energy flow. Insulated segment 94 is of sufficient length to extend into sphincter wall 26 and minimize the transmission of RF energy to a protected site 97 near or adjacent to insulated segment 94 (see FIG. 13). Typical lengths for insulated segment 94 include, but are not limited to, 1-4 mms. Suitable materials for needle electrodes 90 include, but are not limited to, 304 stainless steel and other stainless steels known to those skilled in the art. Suitable materials for insulating layer 92 include, but are not limited to, polyimides and polyamides.

During introduction of treatment apparatus 10, basket assembly 50 is in a contracted state. Once sphincter treatment apparatus 10 is properly positioned at the treatment site 12, needle electrodes 90 are deployed by expansion of basket assembly 50. In one variation of the use of the device to treat a sphincter in the gastrointestinal tract, this results in the protrusion of needle electrodes 90 into the smooth muscle tissue of sphincter wall 26 (refer to FIG. 14). The depth of needle penetration is selectable from a range of 0.5 to 5 mms and is accomplished by indexing movable fitting 82 so as to change the camber 54 of arm 44 in fixed increments that can be selectable in a range from 0.1 to 4 mms. Needle electrodes 90 are coupled to power source 24 via insulated wire 60.

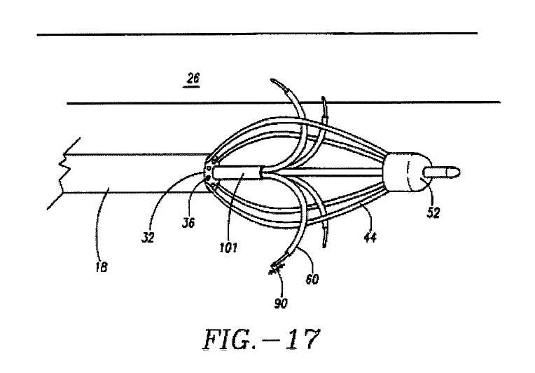

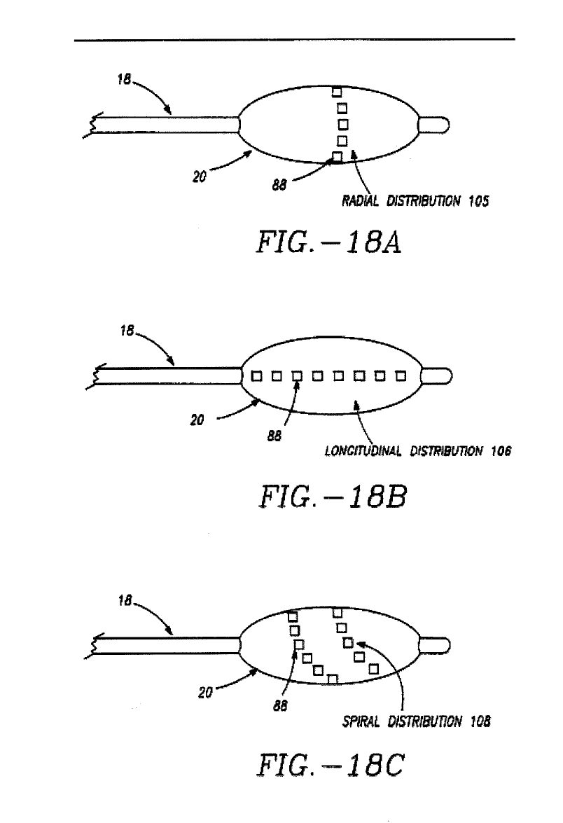

In another embodiment of treatment apparatus 10 shown in FIG. 15, needle electrodes 90 are advanced out of apertures 64 in the body of the device, and/or in basket arms 44. Needle electrodes may be advanced into the target tissue. For example, the needle electrodes may be advanced into smooth muscle of the esophageal wall or other sphincter 16, or the wall of the urethra, or the wall of some other body lumen. In this case, needle electrodes 90 are coupled to RF power source 24 by electrode delivery member 60. In one embodiment, the depth of needle penetration may be selectable via means of stepped sections 66 or tapered sections 68 located in apertures 64. Referring to FIG. 16, apertures 64 and needle electrodes 90 are configured such that the penetration angle 96 (also called an emergence angle 96) of needle electrode 90 into tissue (e.g., sphincter wall 26) remains sufficiently constant during the time needle electrode 90 is being inserted into sphincter wall 26, such that there is no tearing or unnecessary trauma to sphincter wall tissue. This is facilitated by the selection of the following parameters and criteria: i) the emergence angle 96 of apertures 64 which can vary from 1 to 90.degree., ii) the arc radius 98 of the curved section 100 of aperture 64 which can vary from 0.001 to 2 inch, iii) the amount of clearance between the aperture inner diameter 102 and the needle electrode outside diameter 104 which can vary between 0.001″ and 0.1″; and, iv) use of a lubricous coating on electrode delivery member 60 such as a Teflon™ or other coatings well known to those skilled in the art. Also in this embodiment, insulated segment 94 can be in the form of a sleeve that may be adjustably positioned at the exterior of electrode 90.

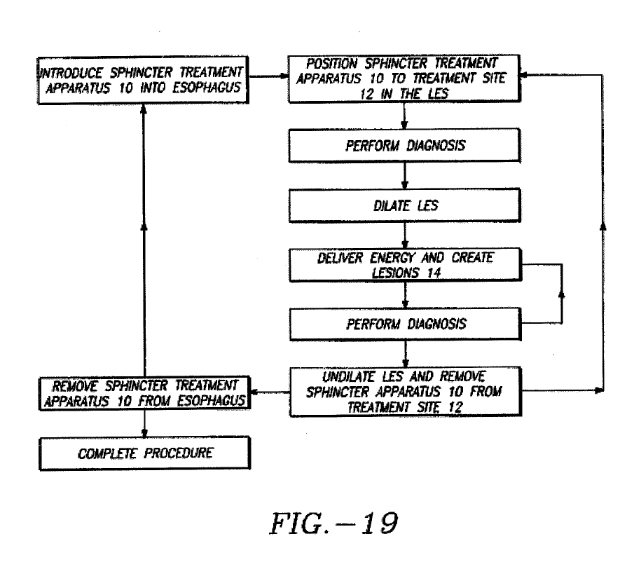

In another alternative embodiment shown in FIG. 17, electrode delivery member 60 with attached needle electrodes 90, can exit from lumen 36 at distal shaft end 32 and be positioned into contact with the patient's tissue 26. This process may be facilitated by use of a hollow guiding member 101, known to those skilled in the art as a guiding catheter, through which electrode delivery member 60 is advanced. Guiding catheter 101 may also include stepped sections 66 or tapered sections 68 at its distal end to control the depth of penetration of needle electrode 90 into sphincter wall 26.

RF energy flowing through tissue causes heating of the tissue due to absorption of the RF energy by the tissue and ohmic heating due to electrical resistance of the tissue. This heating can cause injury to the affected cells and can be substantial enough to denature collagen, shrink cells or support tissues, and even cause cell death, a phenomenon also known as cell necrosis. For ease of discussion for the remainder of this application, cell injury will include all cellular effects resulting from the delivery of energy from electrode 88 up to, and including, cell necrosis. Tissue treatment (including “cell injury”) can be accomplished as a relatively simple medical procedure with local anesthesia. In one embodiment, cell injury proceeds to a depth of approximately 1-4 mms from the surface of the mucosal layer of sphincter 16 or that of an adjoining anatomical structure.

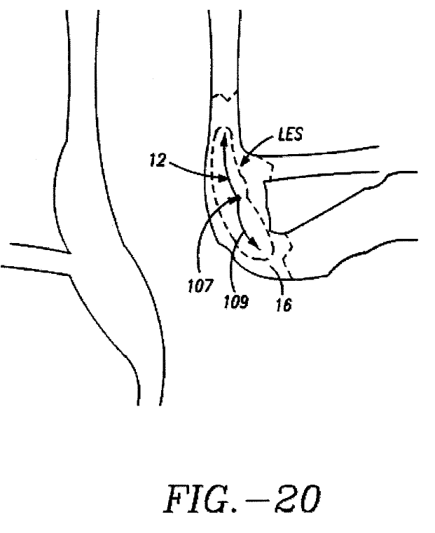

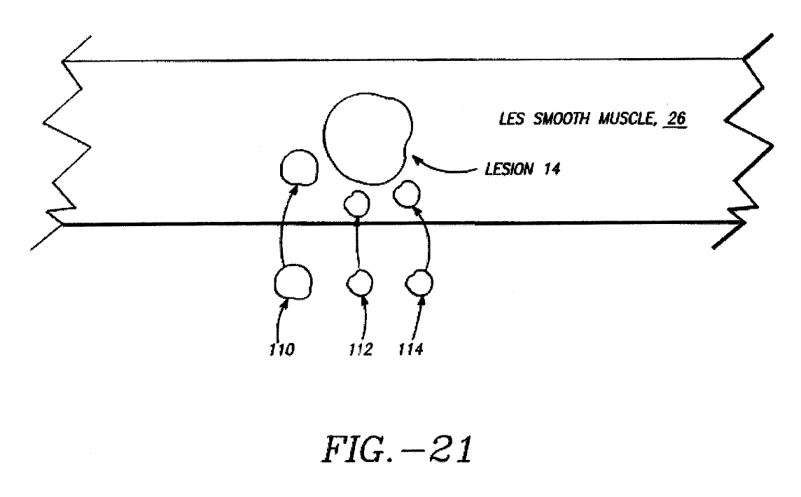

Referring now to FIGS. 18A, 18B and 18C, electrodes 88 and/or apertures 64 may be distributed in a variety of patterns along expansion device 20 or basket assembly 50 in order to produce a desired placement and pattern of lesions 14. Typical electrode and aperture distribution patterns include, but are not limited to, a radial distribution 105 (refer to FIG. 18A) or a longitudinal distribution 106 (refer to FIG. 18B). It will be appreciated that other patterns and geometries for electrode and aperture placement, such as a spiral distribution 108 (refer to FIG. 18C) may also be suitable. These electrodes may be cooled as described hereafter.

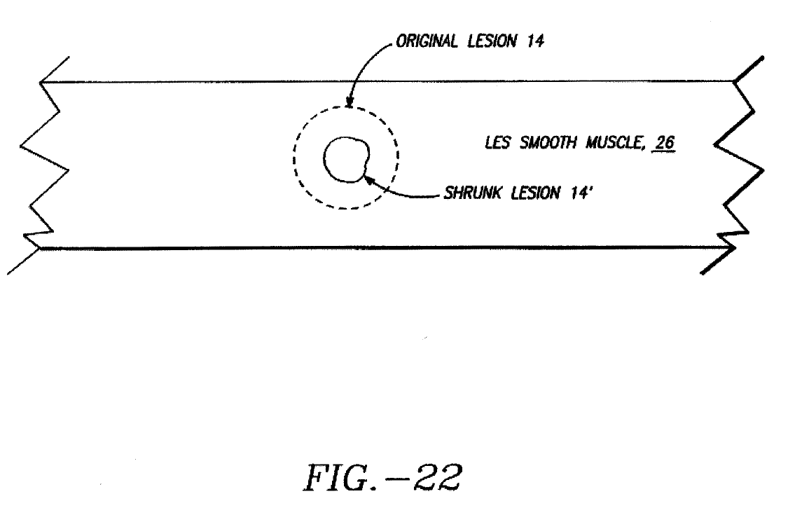

FIG. 19 is a flow chart illustrating one embodiment of the procedure for using treatment apparatus 10. In this embodiment, treatment apparatus 10 is first introduced into the esophagus under local anesthesia. Treatment apparatus 10 can be introduced into the esophagus by itself or through a lumen in an endoscope (not shown), such as disclosed in U.S. Pat. Nos. 5,448,990 and 5,275,608, incorporated herein by reference, or similar esophageal access device known to those skilled in the art. Basket assembly 50 is expanded as described herein. This serves to temporarily dilate the LES or sufficiently to efface a portion of or all of the folds of the LES. In an alternative embodiment, esophageal dilation and subsequent LES fold effacement can be accomplished by insufflation of the esophagus (a known technique) using gas introduced into the esophagus through shaft lumen 36, or an endoscope or similar esophageal access device as described above. Once treatment is completed, basket assembly 50 is returned to its predeployed or contracted state and sphincter treatment apparatus 10 is withdrawn from the esophagus. This results in the LES returning to approximately its pretreatment state and diameter. It will be appreciated that the above procedure is applicable in whole or part to the treatment of other tissues (including other sphincters) in the body.

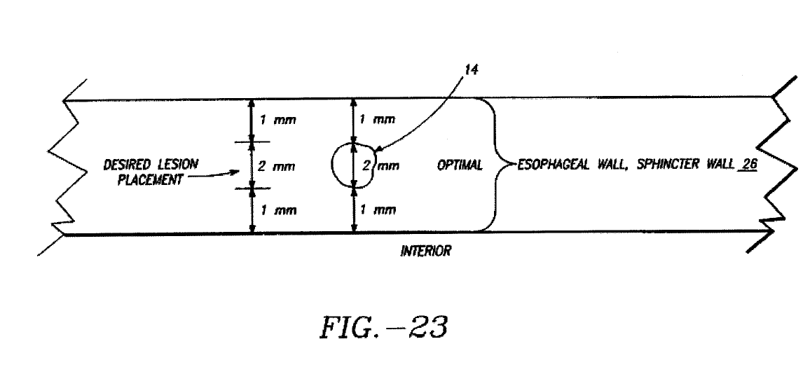

The diagnostic phase of the procedure can be performed using a variety of diagnostic methods, including, but not limited to, the following: (i) visualization of the interior surface of the lumen or tissue wall (e.g., esophagus) via an endoscope or other viewing apparatus inserted into the lumen, (ii) visualization of the interior morphology of the lumen wall using ultrasonography to establish a baseline for the tissue to be treated, (iii) impedance measurement to determine the electrical conductivity between the target tissue and intervening tissues (e.g., in esophageal treatment, the esophageal mucosal layers and sphincter treatment apparatus 10) and (iv) measurement and surface mapping of the electropotential of the lumen (e.g., the LES) during varying time periods which may include such events as depolarization, contraction and repolarization of wall of the lumen (e.g., LES smooth muscle tissue). In some examples, this latter technique is done to determine target treatment sites 12 in the LES or adjoining anatomical structures that are acting as foci 107 or pathways 109 for abnormal or inappropriate polarization and relaxation of the smooth muscle of the LES (Refer to FIG. 20).

In the treatment phase of the procedure, the delivery of energy to treatment site 12 can be conducted under feedback control, manually or by a combination of both. Feedback control (described herein) enables treatment apparatus 10 to be positioned and retained in the body lumen (e.g., urethra or esophagus) during treatment with minimal attention by the physician. Electrodes 88 can be multiplexed in order to treat the entire targeted treatment site 12 or only a portion thereof. Feedback can be included and is achieved by the use of one or more of the following methods: (i) visualization, (ii) impedance measurement, (iii) ultrasonography, (iv) temperature measurement; and, (v) sphincter contractile force measurement via manometry. The feedback mechanism permits the selected on-off switching of different electrodes 88 in a desired pattern, which can be sequential from one electrode 88 to an adjacent electrode 88, or can jump around between non-adjacent electrodes 88. Individual electrodes 88 are multiplexed and volumetrically controlled by a controller.

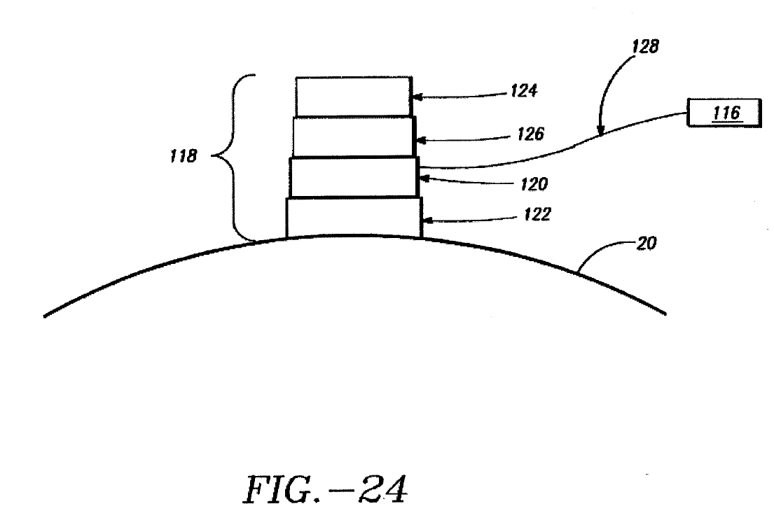

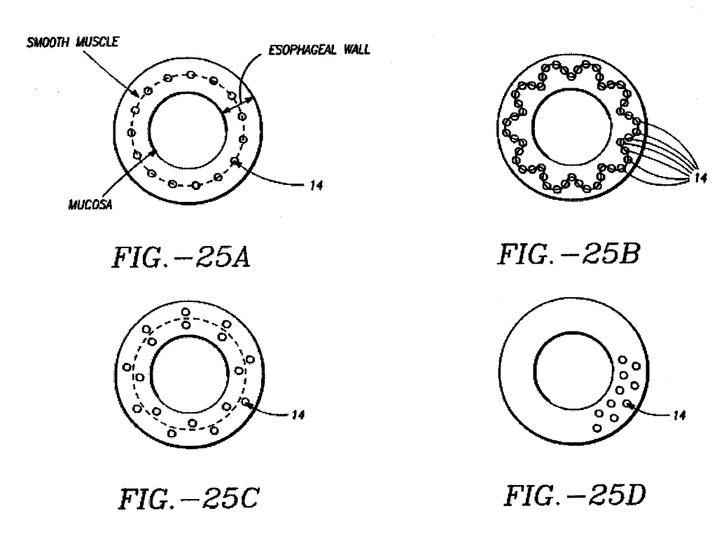

The area and magnitude of cell injury in the target tissue (e.g., LES or sphincter 16) can vary. However, it is desirable to deliver sufficient energy to the targeted treatment site 12 to be able to achieve tissue temperatures in the range of 55-95° C. and produce lesions 14 at depths ranging from 1-4 mms from the interior surface of the body wall (e.g., LES or sphincter wall 26). In one variation, typical energies delivered to the esophageal wall include, but are not limited to, a range between 100 and 50,000 joules per electrode 88. It is also desirable to deliver sufficient energy such that the resulting lesions 14 have a sufficient magnitude and area of cell injury to cause an infiltration of lesion 14 by fibroblasts 110, myofibroblasts 112, macrophages 114 and other cells involved in the tissue healing process (refer to FIG. 21). As shown in FIG. 22, these cells cause a contraction of tissue around lesion 14, decreasing its volume and, or altering the biomechanical properties at lesion 14 so as to result in a tightening of LES or sphincter 16. These changes are reflected in transformed lesion 14′ shown in FIG. 19B. The diameter of lesions 14 can vary between 0.1 to 4 mms. It is preferable that lesions 14 are less than 4 mms in diameter in order to reduce the risk of thermal damage to the mucosal layer. In one embodiment, a 2 mm diameter lesion 14 centered in the wall of the smooth muscle provides a 1 mm buffer zone to prevent damage to the mucosa, submucosa and adventitia, while still allowing for cell infiltration and subsequent sphincter tightening on approximately 50% of the thickness of the wall of the smooth muscle (refer to FIG. 23).

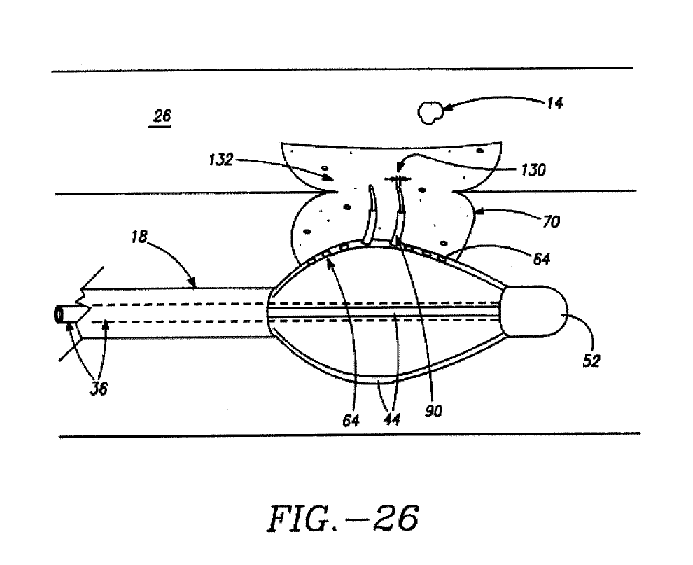

In any of the variations of the devices and methods described herein, it may be beneficial and desirable to image the anatomic area being treated. For example, it may be beneficial to image the interior surface and wall of the body region (e.g., urethra, LES or other sphincter 16, etc.). Imaging may be performed before, during and/or after treatment. The size and position of created treatment regions (e.g., lesions) 14 may be visualized. It may be desirable to create a map of structures (2D or 3D) which can be input to a controller and used to direct the delivery of energy to the treatment site. Referring to FIG. 24, this can be accomplished through the use of ultrasonography (a known procedure) which may involve the use of an ultrasound power source 116 coupled to one or more ultrasound transducers 118 that are positioned on the devices (e.g., on the expansion device 20 or basket assembly 50), or through the use of miniaturized MR technology. An output is associated with ultrasound power source 116.

In general, an imaging modality, including an ultrasound transducer or miniaturized MR, may be incorporated as part of the devices and systems described. For example, as mentioned above, an ultrasound transducer may be used to image the placement location or position of the device. other imaging modalities may also be included, such as fiber-optic (light) based transducers and methods.

For example, an ultrasound transducer 118 can include a piezoelectric crystal 120 mounted on a backing material 122 that is in turn, attached to the device, (e.g., to the expansion device 20 or basket assembly 50). An ultrasound lens 124, fabricated on an electrically insulating material 126, may be mounted over piezoelectric crystal 120. Piezoelectric crystal 120 is connected by electrical leads 128 to ultrasound power source 116. Each ultrasound transducer 118 transmits ultrasound energy into adjacent tissue. Ultrasound transducers 118 can be in the form of an imaging probe such as Model 21362, manufactured and sold by Hewlett Packard Company, Palo Alto, Calif. In one embodiment, two ultrasound transducers 118 are positioned on opposite sides of expansion device 20 or basket assembly 50 to create an image depicting the size and position of lesion 14 in selected sphincter 16.

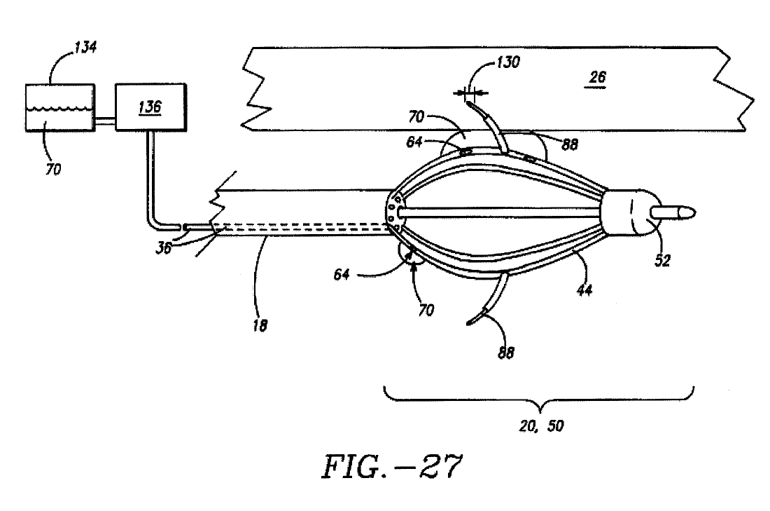

In some variations of the method of treatment described herein for treatment of a sphincter, it is desirable that treatments sites (e.g., lesions) 14 are predominantly located in the smooth muscle layer of selected sphincter 16 at the depths ranging from 1 to 4 mms from the interior surface of sphincter wall 26. However, lesions 14 can vary both in number and position within sphincter wall 26. It may be desirable to produce a pattern of multiple lesions 14 within the sphincter smooth muscle tissue in order to obtain a selected degree of tightening of the LES or other sphincter 16. Typical treatment patterns shown in FIGS. 25A-D include, but are not limited to, (i) a concentric circle of lesions 14 all at fixed depth in the smooth muscle layer evenly spaced along the radial axis of sphincter 16, (ii) a wavy or folded circle of lesions 14 at varying depths in the smooth muscle layer evenly spaced along the radial axis of sphincter 16, (iii) lesions 14 randomly distributed at varying depths in the smooth muscle, but evenly spaced in a radial direction; and, (iv) an eccentric pattern of lesions 14 in one or more radial locations in the smooth muscle wall. Accordingly, the depth of RF and thermal energy penetration sphincter 16 is controlled and selectable. The selective application of energy to sphincter 16 may be the even penetration of RF energy to the entire targeted treatment site 12, a portion of it, or applying different amounts of RF energy to different sites depending on the condition of sphincter 16. If desired, the area of cell injury can be substantially the same for every treatment event. Similarly, when treating tissues of other body lumen, including the urethra, the pattern of treatment sites may be arranged as described above. The pattern may be determined in part by the arrangement of electrodes extending from the treatment apparatus. The treatment device may be moved (e.g., repositioned) to add to or change the pattern of treatment sites.

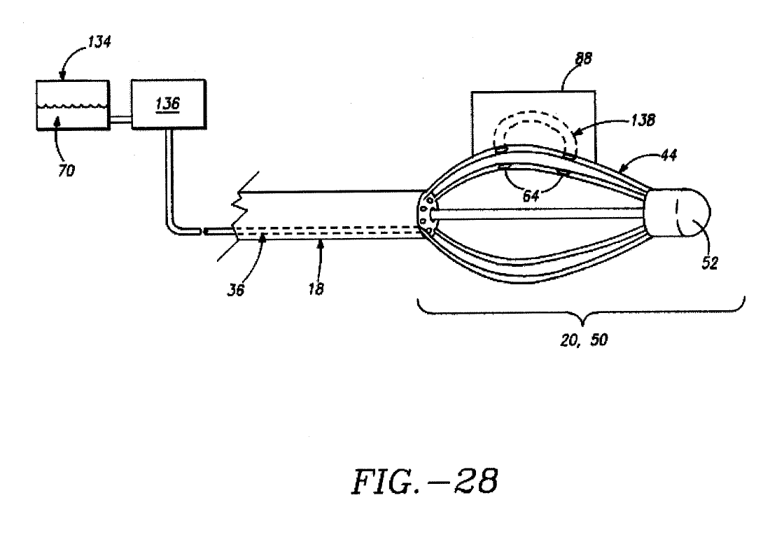

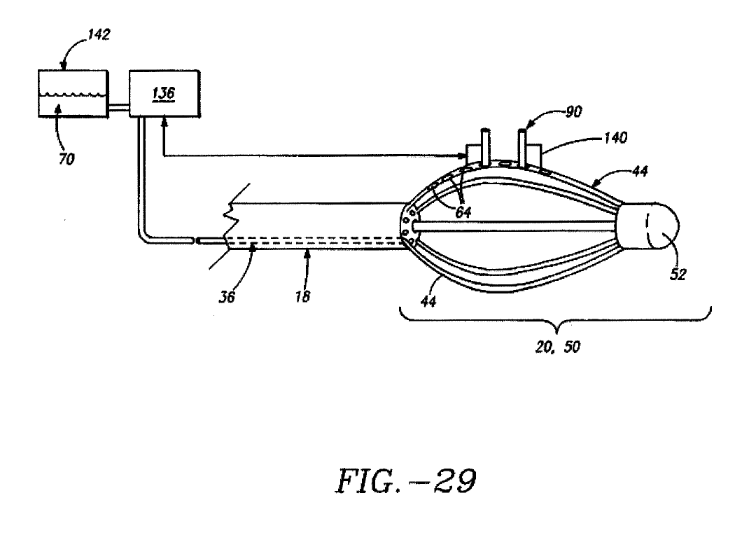

Referring to FIG. 26, it may be desirable to cool all or a portion of the area near the electrode-tissue interface 130 before, during or after the delivery of energy in order to reduce the degree and area of cell injury. For example, the use of cooling preserves the mucosal layers of sphincter wall 26 and protects, or otherwise reduces the degree of cell damage to cooled zone 132 in the vicinity of lesion 14. Referring now to FIG. 27, this can be accomplished through the use of cooling solution 70 that is delivered by apertures 64 which is in fluid communication with shaft lumen 36 that is, in turn, in fluid communication with fluid reservoir 134 and a control unit 136, whose operation is described herein, that controls the delivery of the fluid.

Similarly, it may also be desirable to cool all or a portion of the electrode 88. The rapid delivery of heat through electrode 88, may result in the buildup of charred biological matter on electrode 88 (from contact with tissue and fluids e.g., blood) that impedes the flow of both thermal and electrical energy from electrode 88 to adjacent tissue and causes an electrical impedance rise beyond a cutoff value set on RF power source 24. A similar situation may result from the desiccation of tissue adjacent to electrode 88. Cooling of the electrode 88 can be accomplished by cooling solution 70 that is delivered by apertures 64 as described previously. Referring now to FIG. 28, electrode 88 may also be cooled via a fluid channel 138 in electrode 88 that is in fluid communication with fluid reservoir 134 and control unit 136.

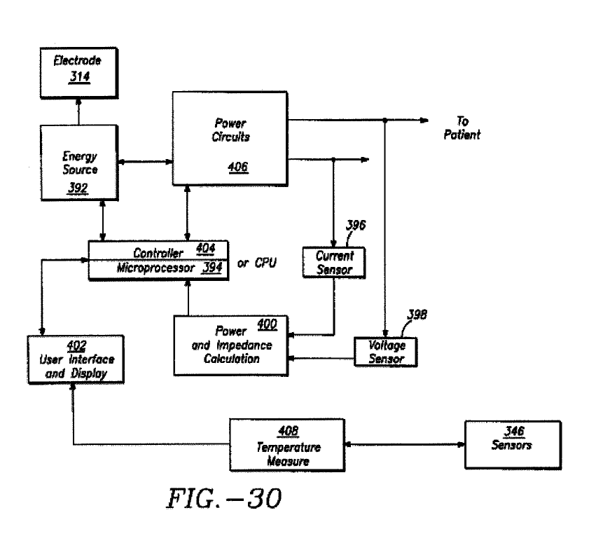

As shown in FIG. 29, one or more sensors 140 may be positioned adjacent to or on electrode 88 for sensing the temperature of sphincter tissue at treatment site 12. More specifically, sensors 140 permit accurate determination of the surface temperature of sphincter wall 26 at electrode-tissue interface 130. This information can be used to regulate both the delivery of energy and cooling solution 70 to the interior surface of sphincter wall 26. In various embodiments, sensors 140 can be positioned at any position on expansion device 20 or basket assembly 50. Suitable sensors that may be used for sensor 140 include: thermocouples, fiber optics, resistive wires, thermocouple IR detectors, and the like. Suitable thermocouples for sensor 140 include: T type with copper constantene, J type, E type and K types as are well known those skilled in the art.

Temperature data from sensors 140 may be fed back to control unit 136 and through an algorithm which is stored within a microprocessor memory of control unit 136. Instructions are sent to an electronically controlled micropump (not shown) to deliver fluid through the fluid lines at the appropriate flow rate and duration to provide control temperature at the electrode-tissue interface 130 (refer to FIG. 27).

The reservoir of control unit 136 may have the ability to control the temperature of the cooling solution 70 by either cooling the fluid or heating the fluid. Alternatively, a fluid reservoir 134 of sufficient size may be used in which the cooling solution 70 is introduced at a temperature at or near that of the normal body temperature. Using a thermally insulated reservoir 142, adequate control of the tissue temperature may be accomplished without need of refrigeration or heating of the cooling solution 70. Cooling solution 70 flow is controlled by control unit 136 or another feedback control system (described herein) to provide temperature control at the electrode-tissue interface 130.

A second diagnostic phase may be included after the treatment is completed. This provides an indication of LES tightening treatment success, and whether or not a second phase of treatment, to all or only a portion of the esophagus, now or at some later time, should be conducted. The second diagnostic phase is accomplished through one or more of the following methods: (i) visualization, (ii) measuring impedance, (iii) ultrasonography, (iv) temperature measurement, or (v) measurement of LES tension and contractile force via manometry.

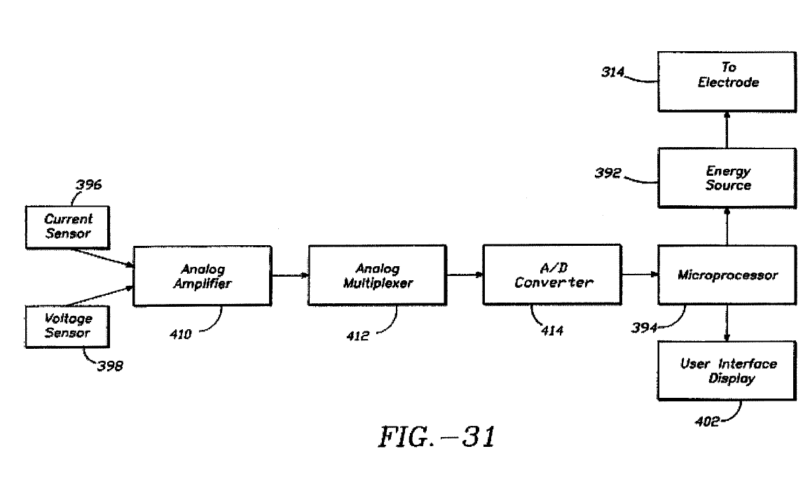

In one embodiment, sphincter treatment apparatus 10 is coupled to an open or closed loop feedback system. Referring now to FIG. 30, an open or closed loop feedback system couples sensor 346 to energy source 392. In this embodiment, electrode 314 is one or more RF electrodes 314.

The temperature of the tissue, or of RF electrode 314 is monitored, and the output power of energy source 392 adjusted accordingly. The physician can, if desired, override the closed or open loop system. A microprocessor 394 can be included and incorporated in the closed or open loop system to switch power on and off, as well as modulate the power. The closed loop system utilizes microprocessor 394 to serve as a controller, monitor the temperature, adjust the RF power, analyze the result, refeed the result, and then modulate the power.

With the use of sensor 346 and the feedback control system a tissue adjacent to RF electrode 314 can be maintained at a desired temperature for a selected period of time without causing a shutdown of the power circuit to electrode 314 due to the development of excessive electrical impedance at electrode 314 or adjacent tissue as is discussed herein. Each RF electrode 314 is connected to resources which generate an independent output. The output maintains a selected energy at RF electrode 314 for a selected length of time.

Current delivered through RF electrode 314 is measured by current sensor 396. Voltage is measured by voltage sensor 398. Impedance and power are then calculated at power and impedance calculation device 400. These values can then be displayed at user interface and display 402. Signals representative of power and impedance values are received by a controller 404.

A control signal is generated by controller 404 that is proportional to the difference between an actual measured value, and a desired value. The control signal is used by power circuits 406 to adjust the power output in an appropriate amount in order to maintain the desired power delivered at respective RF electrodes 314.

In a similar manner, temperatures detected at sensor 346 provide feedback for maintaining a selected power. Temperature at sensor 346 is used as a safety means to interrupt the delivery of energy when maximum pre-set temperatures are exceeded. The actual temperatures are measured at temperature measurement device 408, and the temperatures are displayed at user interface and display 402. A control signal is generated by controller 404 that is proportional to the difference between an actual measured temperature and a desired temperature. The control signal is used by power circuits 406 to adjust the power output in an appropriate amount in order to maintain the desired temperature delivered at the sensor 346. A multiplexer can be included to measure current, voltage and temperature, at the sensor 346, and energy can be delivered to RF electrode 314 in monopolar or bipolar fashion.

Controller 404 can be a digital or analog controller, or a computer with software. When controller 404 is a computer it can include a CPU coupled through a system bus. This system can include a keyboard, a disk drive, or other non-volatile memory systems, a display, and other peripherals, as are known in the art. Also coupled to the bus is a program memory and a data memory.

User interface and display 402 includes operator controls and a display. Controller 404 can be coupled to imaging systems including, but not limited to, ultrasound, CT scanners, X-ray, MRI, mammographic X-ray and the like. Further, direct visualization and tactile imaging can be utilized.

The output of current sensor 396 and voltage sensor 398 are used by controller 404 to maintain a selected power level at RF electrode 314. The amount of RF energy delivered controls the amount of power. A profile of the power delivered to electrode 314 can be incorporated in controller 404 and a preset amount of energy to be delivered may also be profiled.

Circuitry, software and feedback to controller 404 result in process control, the maintenance of the selected power setting which is independent of changes in voltage or current, and is used to change the following process variables: (i) the selected power setting, (ii) the duty cycle (e.g., on-off time), (iii) bipolar or monopolar energy delivery; and, (iv) fluid delivery, including flow rate and pressure. These process variables are controlled and varied, while maintaining the desired delivery of power independent of changes in voltage or current, based on temperatures monitored at sensor 346.

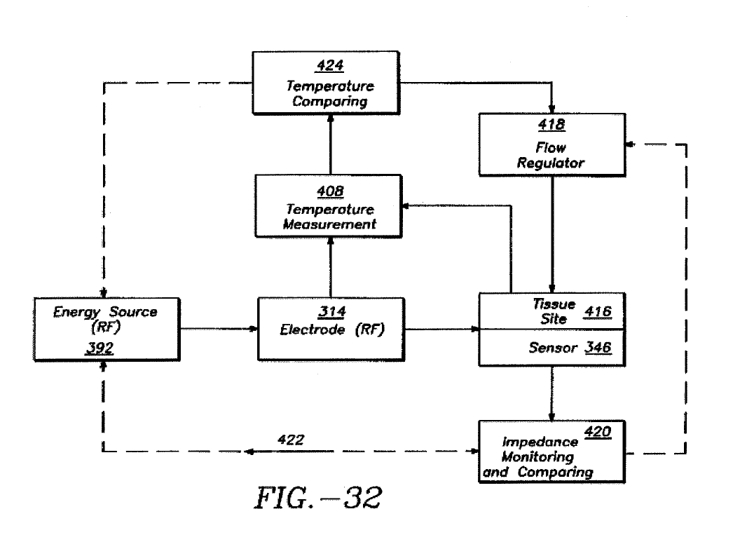

Referring now to FIG. 31, current sensor 396 and voltage sensor 398 are connected to the input of an analog amplifier 410. Analog amplifier 410 can be a conventional differential amplifier circuit for use with sensor 346. The output of analog amplifier 410 is sequentially connected by an analog multiplexer 412 to the input of A/D converter 414. The output of analog amplifier 410 is a voltage which represents the respective sensed temperatures. Digitized amplifier output voltages are supplied by A/D converter 414 to microprocessor 394. Microprocessor 394 may be a type 68HCII available from Motorola. However, it will be appreciated that any suitable microprocessor or general purpose digital or analog computer can be used to calculate impedance or temperature.

Microprocessor 394 sequentially receives and stores digital representations of impedance and temperature. Each digital value received by microprocessor 394 corresponds to different temperatures and impedances.

Calculated power and impedance values can be indicated on user interface and display 402. Alternatively, or in addition to the numerical indication of power or impedance, calculated impedance and power values can be compared by microprocessor 394 to power and impedance limits. When the values exceed predetermined power or impedance values, a warning can be given on user interface and display 402, and additionally, the delivery of RF energy can be reduced, modified or interrupted. A control signal from microprocessor 394 can modify the power level supplied by energy source 392.

FIG. 32 illustrates a block diagram of a temperature and impedance feedback system that can be used to control the delivery of energy to tissue site 416 by energy source 392 and the delivery of cooling solution 70 to electrode 314 and/or tissue site 416 by flow regulator 418. Energy is delivered to RF electrode 314 by energy source 392, and applied to tissue site 416. A monitor 420 ascertains tissue impedance, based on the energy delivered to tissue, and compares the measured impedance value to a set value. If the measured impedance exceeds the set value, a disabling signal 422 is transmitted to energy source 392, ceasing further delivery of energy to RF electrode 314. If measured impedance is within acceptable limits, energy continues to be applied to the tissue.

The control of cooling solution 70 to electrode 314 and/or tissue site 416 is done in the following manner. During the application of energy, temperature measurement device 408 measures the temperature of tissue site 416 and/or RF electrode 314. A comparator 424 receives a signal representative of the measured temperature and compares this value to a pre-set signal representative of the desired temperature. If the tissue temperature is too high, comparator 424 sends a signal to a flow regulator 418 (connected to an electronically controlled micropump, not shown) representing a need for an increased cooling solution flow rate. If the measured temperature has not exceeded the desired temperature, comparator 424 sends a signal to flow regulator 418 to maintain the cooling solution flow rate at its existing level.

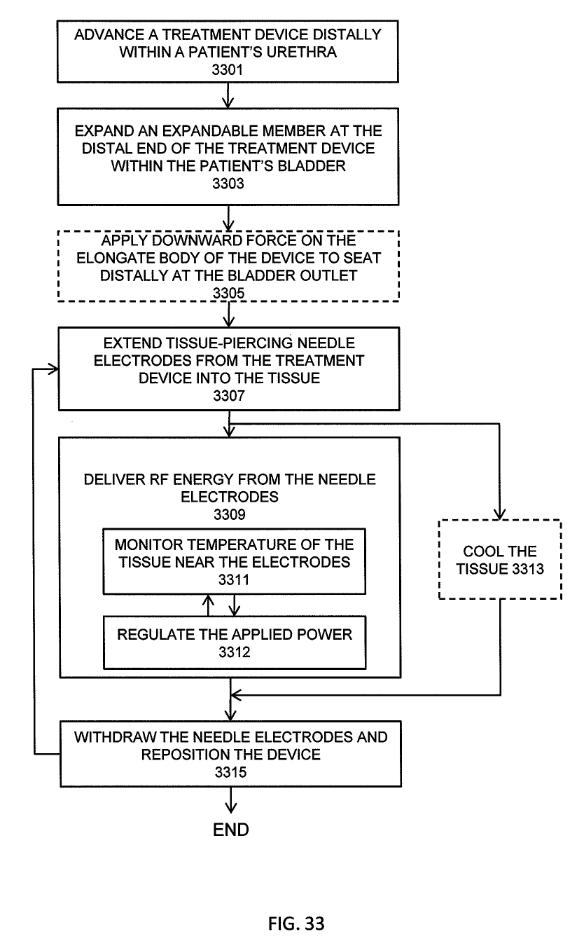

Although the examples illustrated above refer primarily to methods of treating a sphincter, the devices, systems and methods described herein may be used to treat other tissues. In particular, as mentioned above, the devices and systems may be used to treat urinary incontinence by treatment of a subject's urethra. For example, FIG. 33 shows a block diagram of one variation of a method for treating urinary incontinence by applying heat to treat a urethra using the devices described herein. For example, a method of treating a urethra may include delivering energy to a portion of the urethra in an annular pattern of circumferentially spaced-apart treatment sites in the tissue in a patient's urethra by: (1) providing a treatment device having a plurality of tissue-piercing needle electrodes configured to extend from the treatment device and an expandable member at the distal end of the treatment device; (2) advancing the treatment device distally within a patient's urethra 3301; (3) expanding an expandable member at the distal end of the treatment device within the patient's bladder 3303; (4) applying a slight downward force to position the electrodes below the bladder neck 3305, and to seat the expandable region distally at the bladder outlet; (5) extending the tissue-piercing needle electrodes from the treatment device into the tissue 3307; (6) and delivering RF energy from the needle electrodes 3309 resulting in an annular pattern of individual, circumferentially spaced-apart tissue regions. The energy may be applied to a temperature (e.g., between 50° C. and 60° C.) to denature the collagen in the region near the tips of the needle electrodes without causing substantial necrosis. In some variations, the method may also include the step of (7) monitoring the temperature of the tissue near the electrodes 3311 and feeding temperature information back into the controller to regulate the applied power. Fluid (e.g., water) may also be applied to cool the tissue at non-treated (e.g., adjacent) sites 3313 before, during and/or after delivery of energy to treat the tissue. Once this first pass has been completed, the electrodes may be withdrawn, and the device repositioned 3315, and the electrodes extended and powered again 3317, until the desired annular pattern of treatment sites is achieved. In FIG. 33, optional steps are indicated by dashed boxed.

The foregoing description of a preferred embodiment of the invention has been presented for purposes of illustration and description. It is not intended to be exhaustive or to limit the invention to the precise forms disclosed. Obviously, many modifications and variations will be apparent to practitioners skilled in this art. It is intended that the scope of the invention be defined by the following claims and their equivalents.

What is claimed is:

1. A device for delivering energy to a treatment site in an annular pattern of circumferentially spaced-apart regions in the tissue of a body, the device comprising:
an elongate shaft;
an expandable member near the distal end of the elongate shaft configured to secure the treatment device within the body;
a plurality of needle electrodes configured to extend from the device to contact tissue in an annular array that is circumferentially spaced about the axis, each electrode configured to transmit radio frequency energy to the tissue; and
a temperature sensor for sensing the temperature of the tissue at a treatment site.

2. The device of claim 1, further comprising a fluid channel for delivery of a fluid from the device.

3. The device of claim 1, further comprising a fluid channel through each of the needle electrodes configured for delivery of a fluid.

4. The device of claim 1, further comprising a handle at the proximal end of the device configured to manipulate the device.

5. The device of claim 1, wherein the expandable member comprises a basket assembly.

6. The device of claim 1, wherein the expandable member comprises a balloon.

7. The device of claim 1, further comprising a plurality of temperature sensors.

8. The device of claim 1, further comprising a distal cap.

9. A system for delivering energy to a treatment site in an annular pattern of circumferentially spaced-apart regions in the tissue of a body, the system comprising:
a treatment device comprising:
an elongate shaft;
an expandable member near the distal end of the elongate shaft;
a plurality of needle electrodes configured to penetrate the tissue in an annular array that is circumferentially spaced about the axis of the elongate shaft, each electrode configured to transmit radio frequency energy to the tissue; and
a temperature sensor;
a power source configured to provide radio frequency energy to treatment device;
a controller configured to supply radio frequency energy from the power source at to the needle electrodes to treat the tissue in an annular pattern of individual, circumferentially spaced-apart tissue regions, the controller being coupled to the temperature sensor to control the power supplied by the power source to the electrodes.

10. The system of claim 9, wherein the treatment device further comprises a fluid channel for delivery of a fluid from the treatment device.

11. The system of claim 9, wherein the treatment device further comprises a handle at the proximal end of the treatment device configured to manipulate the treatment device.

12. The system of claim 9, wherein the expandable member of the treatment device comprises a basket assembly.

13. The system of claim 9, wherein the expandable member of the treatment device comprises a balloon.

14. The system of claim 9, wherein the plurality of needle electrodes are configured to controllably extend from the shaft.

15. The system of claim 9, wherein the temperature sensor is located adjacent to the needle electrodes and is configured to sense the temperature of the tissue at a treatment site.

16. A method of delivering energy to a tissue in an annular pattern of circumferentially spaced-apart treatment sites in the tissue of a patient's body, the method comprising:
providing a treatment device having a plurality of tissue-piercing needle electrodes configured to extend from the treatment device and an expandable member at the distal end of the treatment device;
advancing the treatment device distally within a lumen in a patient's body to a treatment site;
expanding the expandable member;
extending the tissue-piercing needle electrodes from the treatment device into the tissue; and
delivering RF energy from the needle electrodes in an annular pattern of individual, circumferentially spaced-apart tissue regions.

17. The method of claim 16, further comprising the step of pulling proximally upon the treatment device until resistance to the pulling is encountered before extending the tissue-piercing needle electrodes.

18. The method of claim 16, further comprising introducing a cooling fluid to cool tissue adjacent to the treatment site.

19. The method of claim 16, further comprising sensing a tissue temperature, and controlling delivery of RF energy based, at least in part, upon the sensed tissue temperature condition.

20. The method of claim 16, wherein the step of advancing the treatment device distally comprises advancing the treatment device within a patient's urethra.

21. The method of claim 16, wherein the step of delivering RF energy comprises producing a plurality of submucosal lesions in an annular pattern of individual, circumferentially spaced-apart tissue regions.

22. A method of delivering energy to a tissue in an annular pattern of circumferentially spaced-apart treatment sites in the tissue in a patient's urethra, the method comprising:
providing a treatment device having a plurality of tissue-piercing needle electrodes configured to extend from the treatment device and an expandable member at the distal end of the treatment device;
advancing the treatment device distally within a patient's urethra;
expanding the expandable member within the patient's bladder;
extending at least one tissue-piercing needle electrode from the treatment device into the tissue; and
delivering RF energy from the needle electrodes in an annular pattern of individual, circumferentially spaced-apart tissue regions.
